(19) United States
(12) Patent Application Publication
(10) Pub. No.: US 2005/0138690 A1
(43) Pub. Date: Jun. 23, 2005

(54) Male germ line cell-specific sequences

(76) Inventors: Mohan Singh, Templestowe (AU); Prem Bhalla, Templestowe (AU); Hui-Ling Xu, Doncaster (AU); Ines Swoboda, Vienna (AT); Manjit Singh, Parkville (AU)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/042,574

(22) Filed: Jan. 24, 2005

Related U.S. Application Data
(63) Continuation of application No. 09/463,480, filed on Apr. 7, 2000.

(30) Foreign Application Priority Data
Jul. 25, 1997 (AU) P0 8233
Dec. 31, 1997 (AU) PP 1184

(51) Int. Cl. A01H 1/00; C12N 15/82; C12Q 1/68; C12N 5/04

(52) U.S. Cl. 800/286; 800/287; 435/6; 435/468; 435/419

(57) ABSTRACT

The present invention relates generally to a novel nucleic acid molecule. More particularly, the present invention relates to a male germ line cell specific genetic sequence in plants. Male germ line cells include generative cells and sperm cells. Even more particularly, the present invention provides a male germ line specific gene or functional equivalent thereof. This invention is also directed to the promoter of said gene or its functional derivatives and their use in generating a range of mutant plants including male sterile plants and transposon tagged plants. Preferred nucleic acid molecules of the present invention include the LGC1 gene as well as homologs and derivatives thereof.

### FIELD OF THE INVENTION

The present invention relates generally to a novel nucleic acid molecule. More particularly, the present invention relates to a male germ line cell specific genetic sequence in plants. Male germ line cells include generative cells and sperm cells. Even more particularly, the present invention provides a male germ line specific gene or functional equivalent thereof and to the promoter of said gene or its functional derivatives and there use in generating a range of mutant plants including male sterile plants and transposon tagged plants.

### BACKGROUND OF THE INVENTION

Bibliographic details of the publications numerically referred to in this specification are collected at the end of the description.

The increasing sophistication of recomibinant DNA technology is greatly facilitating research and development in a range of industries and is particularly beneficial for the agricultural and horticultural industries. The ability to manipulate plants and plant products by recombinant means offers great potential to generate relatively quickly new varieties of plants, plants with beneficial genetic alterations and modified plant products, such as grains and fruits.

One important area of the plant industry is the production of hybrid plants. The production of hybrid plants from essentially homozygous parents permits the introduction of a range of beneficial traits including disease resistance, higher seed yield, frost resistance and altered nutritional characteristics.

Due to the importance of hybrid plants to the agricultural and horticultural industries in general, much research has been undertaken to finding improved, more efficacious ways of producing heterozygotic plants. The production of hybrid plants requires that a female parent does not self-fertilize. A range of physical, chemical and genetic techniques have been used or have been proposed in order to prevent self-fertilization. Although some of these techniques have been partially successful, there is still a need to develop alternative, more broadly applicable methods of preventing self-fertilization.

Another important area of the agricultural and horticultural industries is the generation of mutants. Mutant plants may in themselves be useful in removing unwanted traits or may be useful as recipients for further genetic manipulation such as the introduction of new genetic material. Mutant plants have been obtained by a range of procedures including chemical and genetic manipulation as well as physical manipulation and classical breeding. One particularly useful mutant generating mechanism is “transposon tagging”.

Transposons are distinct genetic elements capable of inserting into different sites of the genome within the same cell. Two broad categories of transposons are known comprising the DNA based transposon which transpose via DNA intermediates and retrotransposons or retroelements, which transpose via RNA intermediates. Transposons are useful tools for transposon tagging which relies upon a recognizable phenotype being caused by the insertion into a gene of a transposon. Transposon tagging has found particular application in the cloning of genes.

One system of transposon tagging uses the Activator/Dissociation (Ac/Ds) elements from maize (1). This system comprises a trans-activator, Ac^{st}, which provides a transposase and a cis-responsive Ds element. The transposase promotes high frequency germinal excision of Ds which then reintegrates frequently into new genomic sites after excision.

However, despite the need for male sterile plants and the availability of mutagenic techniques such as transposon tagging, progress has been hampered by the inability to target germ line cells. In work leading up to the present invention, the inventors have identified cDNA clones exhibiting strict generative cell specific expression.

The development of male gametes is one of the most important events in the life cycle of flowering plants. The generative cell, the progenitor of male gametes, plays a central role in this process. This role is to produce two male gametes, the sperm cells, which participate in fertilization. The generative cell residues within the cytoplasm of another cell, the vegetative cell and, until now, was thought to be transcriptionally inactive.

In work leading up to the present invention, the inventors have identified genes which are male gamete specific. The genes and their corresponding promoters of the present invention will enable specific genetic manipulation of the male germ line including generating male sterile plants and facilitating male gamete specific transposon tagging.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Sequence Identity Numbers (SEQ ID NOs.) for the nucleotide and amino acid sequences referred to in the specification are defined following the bibliography.

One aspect of the present invention provides an isolated nucleic acid molecule comprising a nucleotide sequence or a complementary sequence corresponding to a gene or derivative thereof or a region of said gene facilitating its expression wherein said gene is specifically expressed in a male gamete of a plant.

Another aspect of the present invention is directed to a nucleic molecule comprising a nucleotide sequence or complementary sequence encoding an amino acid sequence selected from SEQ ID NO:4, SEQ ID NO:6 and SEQ ID NO:8 or an amino acid sequence having at least 40% similarity to any one of SEQ ID NO:4, SEQ ID NO:6 or SEQ ID NO:8 wherein said nucleic acid molecule exhibits male gamete specific expression in plants.

Another aspect of the present invention provides an isolated nucleic acid molecule comprising a nucleotide sequence or complementary nucleotide sequence selected from the group consisting of SEQ ID NO:3, SEQ ID NO:5 and SEQ ID NO:7 or a nucleotide sequence having at least 50% similarity to any one of SEQ ID NO:3, SEQ ID NO:5 or SEQ ID NO:7 or is a nucleotide sequence capable of hybridizing to any one of SEQ ID NO:3, SEQ ID NO:5 or SEQ ID NO:7 under low stringency conditions at 42° C.

Still yet another aspect of the present invention provides a nucleic acid molecule comprising a promoter or functional derivative thereof which directs plant male gamete specific expression in a nucleotide sequence operably linked thereto.

Even still another aspect of the present invention provides an isolated nucleic acid molecule comprising a nucleotide sequence or complementary nucleotide sequence which is capable of hybridizing under low stringency conditions at 42° C. to a genomic region encompassing at least about 2 kbp upstream of the nucleotide sequence corresponding to any one of SEQ ID NO:3 or SEQ ID NO:5 or SEQ ID NO:7 and wherein said nucleic acid molecule is capable of directing plant male gamete specific expression of a nucleotide sequence operably linked thereto.

Another aspect of the present invention is directed to an isolated nucleic acid molecule comprising a nucleotide sequence or complementary nucleotide sequence substantially as set forth in SEQ ID NO:9 or a nucleotide sequence capable of hybridizing thereto under low stringency conditions at 42° C. or a nucleotide sequence having at least 50% similarity to SEQ ID NO:9 and wherein said molecule is capable of directing plant male gamete specific expression of a nucleotide sequence operably linked thereto.

A further aspect of the present invention contemplates a method of inducing or otherwise facilitating male sterility in a plant, said method comprising operably linking a cytotoxic nucleic acid molecule to a promoter which directs male gamete specific expression in said plant such that upon expression of said promoter, the cytotoxic nucleic acid molecule is expressed to produce a product which inactivates, kills or otherwise renders substantially non-functional male gametes in said plant.

Another aspect of the present invention provides a genetic construct comprising a male gamete specific promoter, as hereinbefore described, operably linked to a transposase gene, said transposase gene capable of inducing transposition of a transposable element, such that upon expression of said promoter, the transposase gene is expressed facilitating transposition of said transposable element.

Reference herein to “male gamete” includes reference to generative cells and sperm cells.

### BRIEF DESCRIPTION OF THE FIGURES

FIG. 1 is a representation of the nucleotide [SEQ ID NO:3] and predicted amino acid [SEQ ID NO:4] sequence of LGC1.

FIG. 2 is a photographic representation showing expression of LGC1 mRNA in different tissues of lily. (A) Northern blot of the indicated tissues probed with ³²P-labelled LGC1 probe. GCs, generative cells. (B) RT-PCR of different tissues. Pollen mRNA includes contribution of both generative cell and vegetative cell. Numbers 16, 31, 64 represent: 1/16, 1/32, and 1/64 of the mRNA input respectively and so forth. Molecular sizes are indicated on the left.

FIG. 3 is a photographic representation showing in situ hybridization of LGC1 mRNA to whole mount lily pollen. Dark staining in the generative cell (arrowhead) represents hybridization signals detected by an alkaline phosphatase conjugated anti-DIG antibody. The outer wall of pollen, exine appears as a sculptured pattern. (A) Pollen probed with a DIG-UTP labelled LGC1 antisense riboprobe. (B) Control pollen probed with a sense riboprobe.

FIG. 4 is a photographic representation showing in situ hybridization of LGC1 mRNA to whole mount lily pollen at different developmental stages. For a better resolution, protoplasts of developing pollen were released from sculptured exine, the outer wall of pollen (9). Developing pollen (A-E) and pollen tube (K) probed with a DIG-UTP labelled riboprobe and then counter-stained with 4′,6′-diamidino-2-phenyl indole (DAPI) to visualize the vegetative and generative nuclei within pollen (F-J) and sperm nuclei in pollen tube (L). Arrowheads indicate the generative cell at early developmental stages. GN, generative nucleus; VN, vegetative nucleus; SC, sperm cell; SN, sperm nucleus.

FIG. 5 is a representation showing nucleotide [SEQ ID NO:5] and deduced amino acid [SEQ ID NO:6] sequences of the gcH2A cDNA. The predicted amino acid sequence (numbered at right) is given below the corresponding nucleic acid sequence (numbered at left).

FIG. 6 is a representation showing nucleotide [SEQ ID NO:7] and deduced amino acid [SEQ ID NO:8] sequences of the Full Length gcH3 cDNA. Numbers at left indicate base positions of the nucleotide sequence, numbers at right residue positions of the derived amino acid sequence.

FIG. 7 is a photographic representation showing expression pattern of gcH2A and gcH3.

FIG. 8 is a photographic representation showing in situ hybridization of gcH2A and gcH3 in pollen. Pollen exine was removed for a better visualising of signal.

(A) Pollen probed with showing strong hybridization signal in the generative cell.

(B) Control pollen probed with DIG-labelled sense gcH2A.

(C) Pollen probed showing strong hybridization signal in the generative cell.

(D) Control pollen probed with DIG-labelled sense gcH3.

FIG. 9 is a photographic representation showing expression of gcH2A and gcH3 during pollen development. In situ hybridization of microspores immediately after formation of generative cell (A, D, G), nearly mature pollen (B, E, H) and mature pollen (C, F, I). Arrow heads indicate nearly formed generative cell VN, vegetative nucleus, GN, generative cell nucleus. Pollen exine was removed for a better visualising of signal.

(A), (B), (C) samples probed with DIG-labelled antisense gcH2A showing strong hybridization signal only in mature pollen.

(G), (H), (I) samples probed with DIG-labelled antisense gcH3 showing hybridization signal only in mature pollen.

(D), (E), (F) DAPI staining of corresponding developmental stages.

FIG. 10 is a representation of the nucleotide sequence of the LGC1 promoter. The transcription start site (nucleotide position 817) and the translation start site (nucleotide position 894) are shown bold and are underlined.

FIG. 11 is a diagrammatic representation showing various constructs comprising the LGC1 promoter, a DNA sequence operably linked thereto and a selectable marker gene (reporter genetic sequence).

FIG. 12(A) is a diagrammatic representation of a genetic construct comprising the LGC1 promoter operably linked to a Gus reporter gene. The genetic construct further comprises a gene conferring a selectable marker.

FIG. 12(B) is a photographic representation showing Gus gene expression using the genetic construct of FIG. 12(A) in mature pollen counterstained with 4′,6′-diamindino-2-phenylindole (DAPI). The observed activity of the LGC1 5′-flanking region thus reflects expression of endogenous LGC1 in lily pollen.

### DETAILED DESCRIPTION OF THE PREFERRED EMBODIMENTS

The present invention provides an isolated nucleic acid molecule comprising a nucleotide sequence or a complementary sequence corresponding to a gene or derivative thereof or a region of said gene facilitating its expression wherein said gene is specifically expressed in a male gamete of a plant. A male gamete is considered to include a vegetative cell and a sperm cell.

The nucleic acid molecule of the present invention extends to a genomic or cDNA molecule corresponding to a gene or its derivative or a promoter of said gene or a functional derivative of said promoter, provided the promoter permits male gamete specific expression of the gene or its derivative.

The plant may be a monocotyledonous or dicotyledonous plant. Preferred plants include but are not limited to legumes, crop, cereal and native grasses, fruiting plants, flowering plants amongst many others. One particularly preferred plant is a lily plant.

In another embodiment, the present invention is directed to a nucleic molecule comprising a nucleotide sequence or complementary sequence encoding an amino acid sequence selected from SEQ ID NO:4, SEQ ID NO:6 and SEQ ID NO:8 or an amino acid sequence having at least 40% similarity to any one of SEQ ID NO:4, SEQ ID NO:6 or SEQ ID NO:8 wherein said nucleic acid molecule exhibits male gamete specific expression in plants. The preferred gene of this aspect of the present invention is referred to as the “LGC1” gene.

Preferably, the percentage similarity is at least about 50%, more preferably at least about 60%, still more preferably at least about 70%, yet even more preferably at least about 80-90% or greater to any one of SEQ ID NO:4, SEQ ID NO:6 or SEQ ID NO:8.

Another aspect of the present invention provides an isolated nucleic acid molecule comprising a nucleotide sequence or complementary nucleotide sequence selected from the group consisting of SEQ ID NO:3, SEQ ID NO:5 and SEQ ID NO:7 or a nucleotide sequence having at least 50% similarity to any one of SEQ ID NO:3, SEQ ID NO:5 or SEQ ID NO:7 or is a nucleotide sequence capable of hybridizing to any one of SEQ ID NO:3, SEQ ID NO:5 or SEQ ID NO:7 under low stringency conditions at 42° C.

Preferably, the percentage level of nucleotide similarity is at least about 60%, more preferably at least about 70%, still more preferably at least about 80%, yet still more preferably at least about 90% or greater to any one of SEQ ID NO:3, SEQ ID NO:5 or SEQ ID NO:7.

Reference herein to a low stringency at 42° C. includes and encompasses from at least about 1% v/v to at least about 15% v/v formamide and from at least about 1M to at least about 2M salt for hybridisation, and at least about 1M to at least about 2M salt for washing conditions. Alternative stringency conditions may be applied where necessary, such as medium stringency, which includes and encompasses from at least about 16% v/v to at least about 30% v/v formamide and from at least about 0.5M to at least about 0.9M salt for hybridisation, and at least about 0.5M to at least about 0.9M salt for washing conditions, or high stringency, which includes and encompasses from at least about 31% v/v to at least about 50% v/v .formamide and from at least about 0.01M to at least about 0.15M salt for hybridisation, and at least about 0.01M to at least about 0.15M salt for washing conditions. In general, washing is carried out Tₘ=69.3+0.41 (G+C) % [19 . However, the Tₘ of a duplex DNA decreases by 1° C. with every increase of 1% in the number of mismatch base pairs (20).

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels.

Preferably, comparisons of nucleotide and amino acid sequences are in terms of percentage identity and this includes the number of exact nucleotide or amino acid matches having regard to an appropriate alignment using a standard algorithm, such as but not limited to the Geneworks programme (Intelligenetics).

Reference to a “derivative” herein includes single or multiple nucleotide or amino acid substitutions, deletions and/or additions as well as parts, fragments, portions, homologues and analogues of the nucleotide or amino acid sequence.

The nucleic acid molecules of the present invention are specifically expressed in male gametes of plants, ie. in the generative cells. The male gamete specific expression is determined in part by the male gamete specific promoter.

Accordingly, another aspect of the present invention provides a nucleic acid molecule comprising a promoter or functional derivative thereof which directs plant male gamete specific expression in a nucleotide sequence operably linked thereto.

More particularly, this aspect of the present invention provides an isolated nucleic acid molecule comprising a nucleotide sequence or complementary nucleotide sequence which is capable of hybridizing under low stringency conditions at 42° C. to a genomic region encompassing at least about 2 kbp upstream of the nucleotide sequence corresponding to any one of SEQ ID NO:3 or SEQ ID NO:5 or SEQ ID NO:7 and wherein said nucleic acid molecule is capable of directing plant male gamete specific expression of a nucleotide sequence operably linked thereto.

Even more particularly, this aspect of the present invention is directed to an isolated nucleic acid molecule comprising a nucleotide sequence or complementary nucleotide sequence substantially as set forth in SEQ ID NO:9 or a nucleotide sequence capable of hybridizing thereto under low stringency conditions at 42° C. or a nucleotide sequence having at least 50% similarity to SEQ ID NO:9 and wherein said molecule is capable of directing plant male gamete specific expression of a nucleotide sequence operably linked thereto.

The nucleotide sequence of SEQ ID NO:9 represents the promoter of the LGC1 gene and is referred to herein as the LGC1 promoter. The present invention encompasses the LGC1 promoter comprising a nucleotide sequence substantially as set forth in SEQ ID NO:9 or any derivative thereof which includes mutants, fragments, homologues and analogues thereof. Such derivatives are conveniently further defined by being able to hybridize under low stringency conditions at 42° C. to SEQ ID NO:9 and/or have a nucleotide sequence of about 50% similarity to SEQ ID NO:9. Generally, the derivatives retain at least partial promoter activity and, hence, are “functional” derivatives. However, non-functional derivatives are also encompassed by the present invention since these have utility, for example, in inhibiting promoter activity and as probes for other similar promoters.

In SEQ ID NO:9, the transcription start site is at nucleotide position 817 and the translation start site (ATG) is at nucleotide position 894.

The present invention further extends to a variety of genetic constructs comprising the LGC1 promoter or its derivatives together with a nucleotide sequence operably linked to the promoter and optionally a report molecule. Examples of nucleotide sequences operably linked to the promoter include, but are not limited to, those encoding GUS, GFP, ribonuclease, DTA, antisense molecules, transposons, ribozymes and lethal genes amongst many others.

The identification of a male gamete specific promoter and gene permits the generation of a range of male sterile plants as well as male gamete specific transposon tagging.

In one embodiment, the present invention contemplates a method of inducing or otherwise facilitating male sterility in a plant, said method comprising operably linking a cytotoxic nucleic acid molecule to a promoter which directs male garnete specific expression in said plant such that upon expression of said promoter, the cytotoxic nucleic acid molecule is expressed to produce a product which inactivates, kills or otherwise renders substantially non-functional male gametes in said plant.

The cytotoxic nucleic acid molecule may encode or comprise a cytotoxic protein, an antisense molecule to a particular gene, a ribozyme or a plantabody amongst many other molecules.

Preferably, the promoter corresponds to a nucleotide sequence which hybridizes under low stringency conditions to a genomic region comprising at least about 2 kbp upstream of a gene corresponding to any one of SEQ ID NO:3, SEQ ID NO:5 or SEQ ID NO:7. More particularly, the promoter is the LGC1 promoter or its derivatives.

Alternatively, the cytotoxic nucleic acid molecule is fused to the gene naturally operably linked to said promoter such that upon expression of said gene, the cytotoxic nucleic acid molecule inactivates, kills or otherwise renders substantially non-function a male gamete in said plant.

In another embodiment, the male gamete specific promoter and/or gene is used to facilitate male gamete specific transposon tagging. This facilitates the product of pollen grains in a plant carrying a transposon tag. Offspring can then be screened for a range of phenotypes of interest and then, in turn, the transponson tagged plants used to clone particular genes.

Accordingly, another aspect of the present invention provides a genetic construct comprising a male gamete specific promoter, as hereinbefore described, operably linked to a transposase gene, said transposase gene capable of inducing transposition of a transposable element, such that upon expression of said promoter, the transposase gene is expressed facilitating transposition of said transposable element.

A particularly useful transposon system is the Ds^{ALS} system (1, 5) where the activator (Ac) transposase would be under the control of the promoter of the present invention to facilitate transposition of the dissociation (Ds) element.

In accordance with the present invention a plant is selected such as a crop plant, legume, grass plant or flowering plant amongst other monocots and dicots and a callus culture prepared. A genetic construct comprising the male gamete specific promoter and optionally male gene specific gene naturally associated with said promoter operably linked to a cytotoxis nucleic acid molecule or a transposase gene is introduced into callus cells. A plant is then regenerated. The male gamete specific construct may be under additional control mechanisms such as environmental, developmental, physiological or nutritional control mechanisms such that upon provision of these mechanisms, the male gamete specific promoter is activated. In any event, upon expression of the male gamete specific promoter, transposon tagging will occur or the cytotoxic nucleic acid will be expressed. This will result in tagged pollen or male sterility.

Male sterile plants containing a range of transposon insertions and genetic constructs useful of the practice of the present invention are all encompassed by the present invention as are all offspring or progeny, new plant varieties and mutant plants.

The present invention extends to the promoter as herein described as well as functional mutants thereof. A functional mutant includes promoter fusions to other promoters, as well as single or multiple nucleotides, deletions, additions and/or substitutions including parts, fragments, portions, homologues and analogues thereof.

Although not intending to limit the present invention to any one type of male gamete specific gene or promoter, genes and their promoters encoding histones are particularly useful.

Another benefit of the present invention provides the potential to develop seedless fruit or fruit with reduced seed content. This is particularly applicable where pollination stimulates fruit development and where the lack of fertilization results in seedless fruit.

The present invention extends to any transposable element such as but not limited to Ac, Ds, En/Spm, dspm, Tam3, dTam3, Mu1, Tat1, Tag1, dTph1, Tnt1, Tto1, Tto2, Ac-like, dTnp and Tos17. These elements are conveniently reviewed in the reference (16).

The present invention is further described by the following non-limiting Examples.

### EXAMPLE 1

### Isolation of LGC1

Generative cells from lily (Lilium longiflorum) were isolated and mRNA isolated therefrom. Generative cells were isolated from fresh pollen of lily as previously described (6) and stored at −70° C. until use. mRNA was extracted directly from approximately 1×10⁵ of stored generative cells using a mRNA purification kit (Pharmacia-LKB). Purified generative cell mRNA was reverse transcribed and the resultant cDNA was amplified by PCR, size fractionated and cloned into λgt11 expression vector.

A differential hybridization approach was used to obtain a cDNA clone corresponding to a gene specifically expressed in generative cells. The clone was designated LGC1. In the differential hybridization approach, a number of cDNA clones were randomly picked from a generative cell cDNA library and cDNA inserts obtained by PCR with λgt11 forward and reverse primers. PCR conditions were 30 cycles of 1 min at 94° C., 2 min at 60° C. and 3 min at 72° C. extension at 72° C. for 10 min. The amplified cDNA inserts were purified, labelled with ³²P by random priming (Bresatec Ltd, South Australia) and used for probing of RNA slot blots containing approximately 300 ng of mRNAs from various tissues including leaf, stem, petal, stigma/style, ovary, pollen and generative cells. Hybridization and washing was performed as previously described (18). cDNA clones showing preferential or specific hybridization to generative cell mRNA were selected for further analysis.

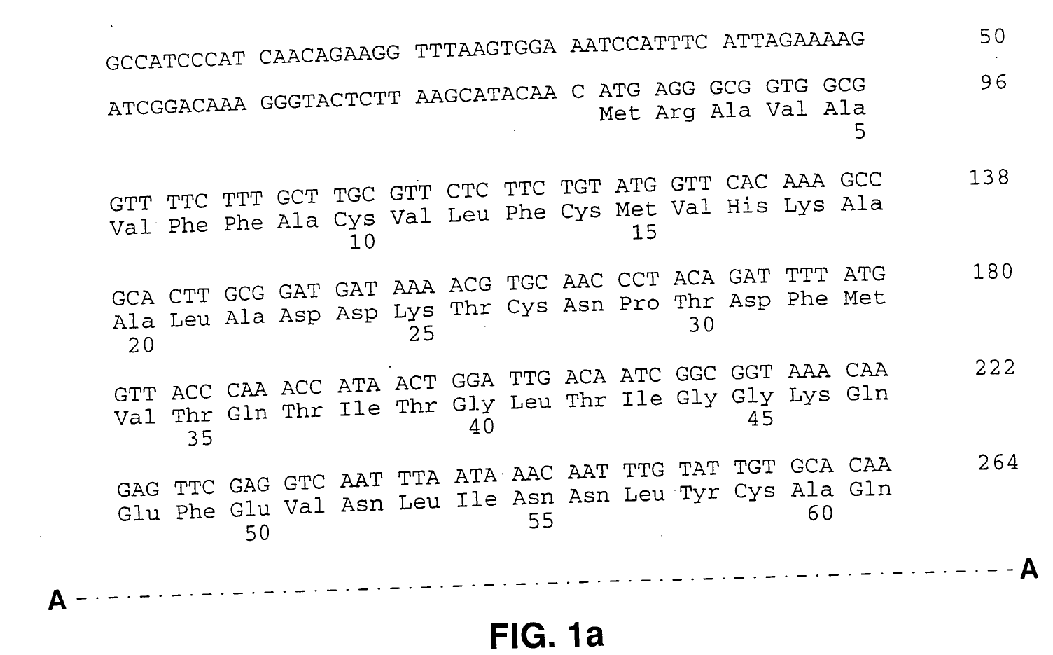

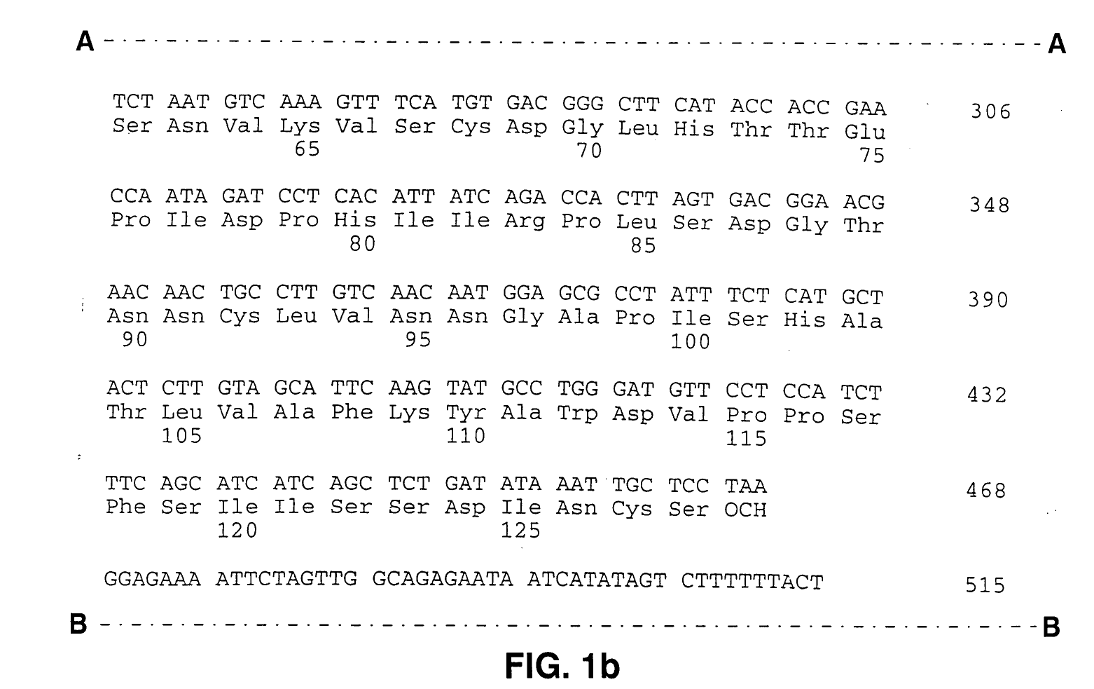

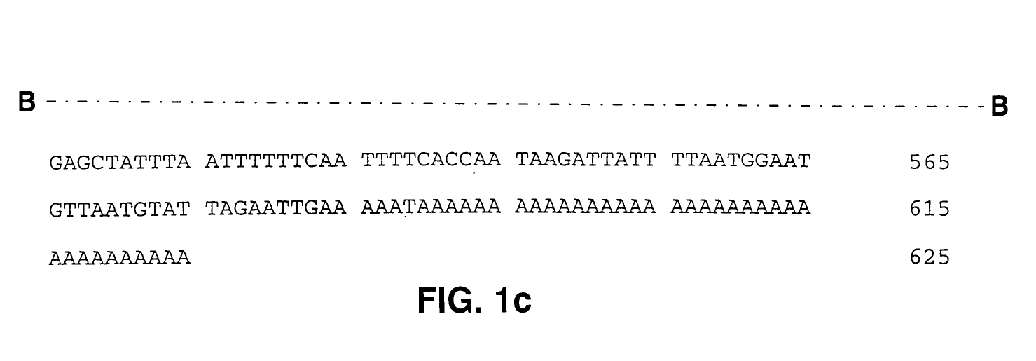

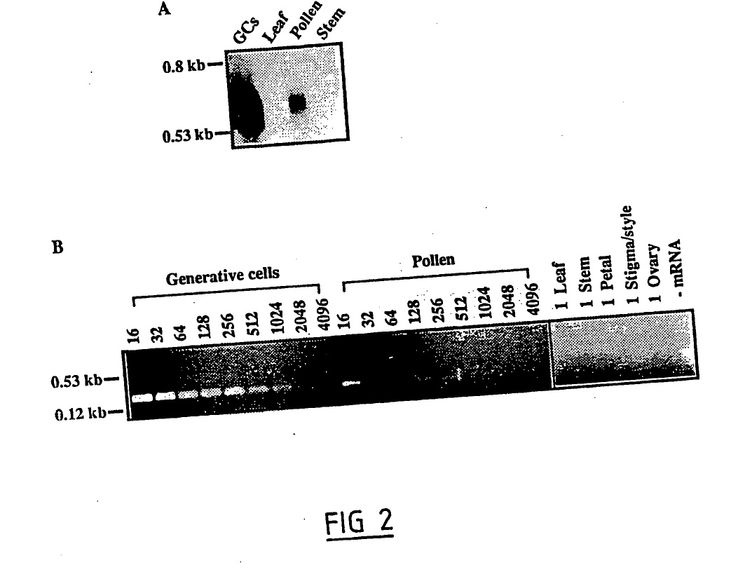

The cDNA insert of one clone, LGC1, was subcloned into pBluescript(SK)+(Stratagene) and sequenced with ABI PRISM (trademark) dye terminator cycle sequencing kit (Perkin-Elmer). The LGC1 cDNA insert was shown to be 618 bp in length encoding a predicted gene product of 128 amino acids with a calculated molecular weight of 13.8 kDa (FIG. 1). LGC1 corresponds to a 0.6 kbp transcript which is present at a high level in generative cells as revealed by Northern blot analysis (FIG. 2A).

No signal was detectable in the two vegetative tissues tested, leaf and stem, while a faint signal was visible in pollen containing generative cells. The tissue specificity of LGC1 was further examined by RT-PCR using gene specific PCR primers that amplify a 0.3 kbp portion of the coding region. For RT-PCR, mRNAs from generative cells and various tissues were reverse transcribed and amplified by PCR with a pair of sequence specific primers (L13A: 5′-GTACTCTTAAGCATACAACATGAG-3′ [SEQ ID NO:1]; L13B: 5′-CAGGCATACTTGAATGCTACAAGA-3′ [SEQ ID NO:2]) using the Access RT-PCR System (Promega). For each tissue, mRNA was subjected to a serial two-fold dilutions. Based on the signal intensity of the amplified products, the relative amount of LGC1 mRNA in each tissue was estimated.

RT-PCR amplifications were performed using controlled amount of RNA input from various tissues of lily plant. A PCR product of expected size (0.3 kbp) was obtained in generative cells and pollen but not in all the other tissues tested including vegetative parts such as leaf, stem as well as reproductive parts such as petal, female stigma/style and ovary (FIG. 2B). Based on the signal intensity, the inventors estimated that approximately 20 fold more PCR product was obtained when generative cell mRNA was used as compared to pollen mRNA. Since the generative cell constitutes a small portion of pollen, the inventors considered that the amplified LGC1 product obtained using pollen mRNA input may represent the contribution of generative cell only. Generative cell specificity of LGC1 was further confirmed by in situ hybridization as hereinafter described.

Non-radioactive whole mount in situ hybridization was performed in both developing and mature pollen based on the protocols previously described (3, 4, 5). Fresh pollen at various developmental stages was fixed (1% v/v glutaraldehyde in 50 mM PIPES buffer, pH 7.4) for 2 hours at room temperature. The fixed pollen was then washed in buffer and stored in 70% v/v ethanol at 4° C. until use. Both sense and antisense riboprobes labelled with DIG-UTP were generated from linearized DNA templates. The hybridization signal was detected with an alkaline phosphatase conjugated anti-DIG antibody using a DIG nucleic acid detection kit (Boehringer Mannheim). To obtain a better resolution, protoplasts of developing pollen were released from exine (the outer wall of pollen) by treatment with enzyme solution (1% w/v Macerozyme, 0.5% w/v Cellulase and 0.5% w/v BSA) as previously described (6). Vegetative and generative nuclei within pollen were visualized by counter-staining with 4′,6′-diamindino-2-phenyl indole (DAPI).

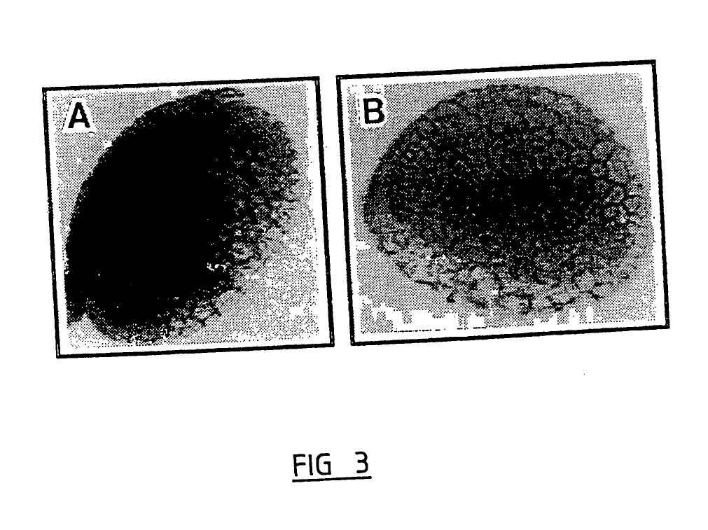

The results clearly showed that LGC1 mRNA is confined to the generative cell in mature pollen (FIG. 3). LGC1 mRNA in pollen as detected by Northern blot and RT-PCR own their origin to the generative cell.

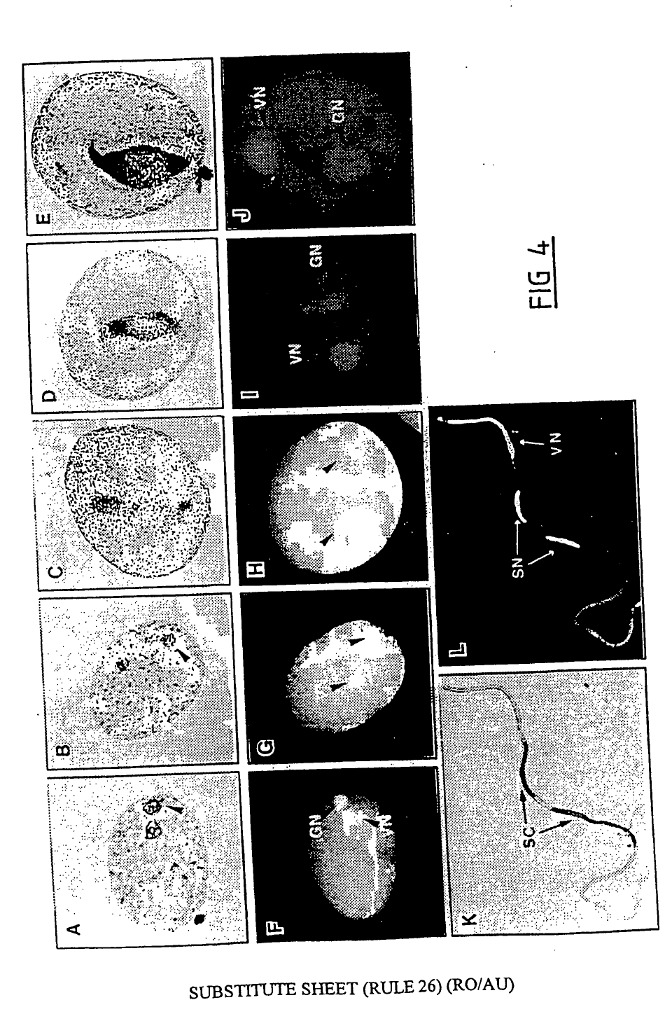

To determine whether LGC1 mRNA present in the generative cell is the product of generative cell specific gene activity or the result of asymmetric RNA localization and partitioning prior to generative cell formation in developing pollen, the inventors monitored LGC1 mRNA accumulation during this process. The inventors examined six different developmental stages of generative cells. At the early stage, the newly formed generative cell is attached at one pole of pollen with the vegetative nucleus located in its vicinity (FIGS. 4A, F). As the development progresses, the generative cell starts to detach itself from the intine (inner cell wall of pollen) while the vegetative nucleus moves towards the centre of pollen (FIGS. 4B, G). No detectable signal was observed in these two early developmental stages (FIGS. 4A, B). With rapid size expansion of pollen, the generative cell separates completely from the intine and suspends freely within the vegetative cell cytoplasm. At this stage, its shape becomes elongated with a large nucleus in the centre and most of cytoplasm at both ends of the cell (FIGS. 4C, H). A weak signal was detected at both ends of the generative cell, indicating the initiation of LGC1 mRNA transcription (FIGS. 4C). As the development continues, the generative cell becomes spindle-shaped (FIGS. 4D, I) and accumulation of LGC1 mRNA in the generative cell becomes more evident (FIGS. 4D). At the time of pollen maturity, a very high level of LGC1 mRNA were observed in the generative cell (FIG. 3A, FIGS. 4E, J). Next, pollen germination occurs on female stigma and pollen tubes grow inside the female stylar tissue. The generative cell then moves into pollen tube and undergoes a mitotic division producing two male gametes, the sperm cells (FIGS. 4K, L). LGC1 mRNA was clearly detectable in the two sperm cells inside the pollen tubes (FIG. 4K) as described more fully below.

In lily, generative cell division occurs in the pollen tube during its growth in the female stylar tissue. In situ hybridization of mRNA in sperm cells, therefore, can only be performed in pollen tube. Pollen tubes were grown in vivo by hand pollinating pistils with freshly collected pollen.

After 48 hours, a 1 cm long segment was taken from the base of the style and cut into two symmetrical halves. Pollen tubes growing in the hollow stylar canal were teased out, fixed and then used for in situ hybridization as described above.

No signal was detected in the vegetative cell at any stage of pollen development. These results show that the generative cell specific accumulation of LGC1 mRNA is due to differential gene activation of generative cell.

Male germ line specific gene expression represents a new aspect of fundamental importance in flowering plants. LGC1 is the first male germ line specific gene to be identified in flowering plants and thus, the present study of generative cell specific gene expression has important implications in understanding the molecular bases of male gamete development. Several aspects of research can immediately benefit from the availability of this gene and its promoter. For example, selective ablation of the male gametes can be achieved using generative cell specific promoter-cytotoxin fusions. The availability of LGC1 gene promoter will make it possible to introduce marker genes for monitoring the process of sperm-egg recognition and fusion at molecular level. Furthermore, the male gamete specific promoter may be used to generate a range of transposos to specify tagged pollen genes.

### EXAMPLE 2

### Male Gamete Cell Specific Expression of H2A and H3 Histone Genes

The following Examples shows the identification of two cDNA clones, gcH2A and gcH3, which encode male gamete-specific variants of histones H2A and H3, respectively. The inventors show that both gcH2A and gcH3 mRNAs accumulate exclusively within the male germ line cell, the generative cell. An examination of the spatial distribution of gcH2A and gcH3 transcripts during pollen development show that initiation of expression of these genes occurs in generative cell at the later stages of pollen maturation. The results indicate that these histone variants are the products of generative cell transcriptional activity. This example provides the first insight of male germ line cell specific histone gene expression in flowering plants.

1. Introduction

Histones are the major protein constituents of the chromatin of eukaryotic cell nuclei. Histone proteins include five major classes: four core histones, H2A, H2B, H3, H4 and one linker histone H1. The core histones are small, basic proteins (11-15 kDa) that contain a high proportion of positively charged amino acids, mainly lysine and arginine. Histones are highly conserved throughout evolution and are encoded by multigene families. Genes encoding major classes of histones are usually expressed in a cell cycle-dependent fashion at the beginning of the S (DNA synthesis) phase and are coordinately regulated at the transcriptional and post-transcriptional level through the cell cycle (7).

2. Methods

(a) Construction and Screening of cDNA Library

Generative cells were isolated from mature pollen of lily (Lilium longiflorum) as previously described (8) and stored at −70° C. until use. Poly(A)+ RNA was isolated from approximately 1×10⁵ of stored generative cells using oligo (dT)-cellulose affinity column (Pharmacia) according to the manufacture's instruction. First-strand cDNA was synthesized with an oligo (dT) primer. A Capswitch primer was also used to ensure the synthesis of full length clones. The resultant cDNA was amplified by PCR using the following conditions: 35 cycles of 94° C. for 1 min, 42° C. for 2 min and 72° C. for 2 min. The PCR products were size-fractionated through a Sephadex-50 column and cDNAs of appropriate size were cloned into λgt11 expression vector.

For screening, a number of cDNA clones was randomly picked and cDNA inserts were obtained by PCR with λgt11 forward and reverse primers. Differential screening was conducted by probing RNA slot blots of various tissues with the amplified cDNA inserts. cDNA clones showing strong hybridization to generative cell RNA, weak hybridization to pollen RNA and no hybridization to other tissues were considered to be putative generative cell-specific clones.

(b) Sequencing Analysis

The putative generative cell cDNA clones were subcloned into pBluescript II SK+ (Stratagene). Sequencing was performed on both strands by the dideoxy chain-termination method (9) using ABI PRISM (trademark) dye terminator cycle sequencing kit (Perkin-Elmer) with an automated DNA sequencer. Sequence-specific primers were used to generate overlapping sequence information. DNA and protein sequence analysis was performed using BLAST search tools.

(c) RNA Gel Blot Analyses

Total RNA was prepared from various tissues (10). Generative cell RNA was isolated using SNAP RNA extraction kit (Invitro Gene) according to the manufacture's procedure. For gel blot analysis, 20 μg of total RNA was separated by denatured agarose gel electrophoresis, blotted onto Hybond N+ nylon membrane (Amersham) and probed with ³²P-labelled gcH2A and gcH3 cDNA inserts. Hybridization of probes with RNA blots was performed in 50% v/v deionised formamide, 2×SSPE (1×SSPE is 0.15 M NaCl, 0.01 M NaH₂PO₄, and 1 mM EDTA, pH 7.4), 1% w/v PEG, 0.5% w/v BLOTTO, 7% w/v SDS and 0.5 mg/ml denatured salmon sperm DNA at 42° C. overnight. The blots were washed with 2×SSC (1×SSC is 0.15 M NaCl and 15 mM sodium citrate, pH 7.0), 0.1% w/v SDS at room temperature for 15 min and with 0.2×SSC, 0.1% w/v SDS at 65° C. for 15 min, followed by a brief wash in 0.2×SSC. The blots were re-probed with lily ribosome RNA to verify the relative amount of RNAs loaded.

(d) In situ Hybridization

Non-radioactive whole mount in situ hybridization was performed based on the protocols described (11, 12, 13). Developmental stages of pollen were determined using 4′,6′-diamidino-2-phenyl indole (DAPI) staining. Mature and developing pollen was treated with an enzyme solution (1% wlv macerozyme, 0.5% w/v cellulase and 0.5% w/v BSA) for 1 hour to remove the exine (the outer wall of pollen). Pollen protoplasts were then washed in 50 mM PIPES buffer and fixed in 1% v/v glutaraldehyde in 50 mM PIPES buffer, pH 7.4, for 2 hours at room temperature. The fixed pollen was then washed in 50 mM PIPES buffer and stored in 70% v/v ethanol at 4° C.

Prior to hybridization, pollen samples were first dehydrated through an ethanol series up to 100% v/v ethanol. Samples were then treated with xylene (2×10 min) followed by rehydration through an ethanol series. Proteinase K (1 μg/ml) treatment was carried out in 100 mM Tris-HCl, pH 8 and 50 mM EDTA for 40 min at 37° C. Digoxigenin-labelled riboprobes were synthesized by in vitro transcription (Promega). Hybridization was performed in 50% v/v formamide, 6×SSC, 3% w/v SDS, 100 μg/ml tRNA at 55° C. overnight. Samples were then washed in 1×SSC, 0. 1% w/v SDS at room temperature followed by 2×10 min washes in 0.2 SSC, 0.1% w/v SDS at 55° C. RNase A (10 μg/ml) treatment was performed in 2×SSC for 1 hour at 37° C. Hybridization signal was detected using a DIG detection kit (Boehringer Mannheim) according to the manufacture's specification. Vegetative and generative cell nuclei were visualized by counter-staining with DAPI.

Results

Isolation and Characterisation of Histone gcH2A and gcH3 cDNA Clones

Lily (Lilum longiflorum) was used as an experimental system in accordance with the present Example. Within the pollen grain, the male germ line cell (generative cell) is enclosed in the much larger vegetative cell. To maximize the chance of obtaining genes specifically expressed in the generative cell, the inventors prepared a cDNA library using polyA(+) RNA from isolated generative cells. The cDNA library was screened by differential hybridization using probes from generative cells, pollen, leaf, stem, pistil and ovary. cDNA clones that gave strong positive hybridization signal with generative cell mRNA, weak signal with pollen mRNA and no signal with mRNA from other tissues were considered as putative generative cell specific clones. These cDNA clones were subjected to further analysis. Two of these clones were found to encode proteins which were identified as variants of histone H2A and H3, respectively. The two clones were designated “gcH2A” and “gcH3”.

gcH2A cDNA is 581 bp long and contains an open reading frame of 333 bp starting from the first ATG at position 49 to a stop codon TAA at position 379 (FIG. 1). The derived amino acid sequence of gcH2A is composed of 111 amino acids and encodes a protein with a calculated molecular mass of 12.1 kDa gcH2A polypeptide contains 10.8% arginine and 5.4% lysine. The deduced amino acid sequence of gcH2A shows high levels of sequence similarity as well as variability when compared to somatic H2A histones from other organisms. The N-terminal region of the protein appeared to be more conserved than the C-terminal region. In addition, gcH2A polypeptide is 30-35 amino acids shorter at the C-terminus than somatic H2A histone. It has been reported that the C-terminal variable regions of wheat somatic histones can be of two structural different types (14). Type 1 H2A proteins have one or two copies of a SPKK motif which is known to interact with the minor groove of the DNA, whereas type 2 H2A proteins have a shorter C-terminal variable region and no SPKK motif. Using these criteria, the lily generative cell specific H2A (gcH2A) histone can be classified as type 2 since the C-terminal region of gcH2A does not contain a SPKK motif.

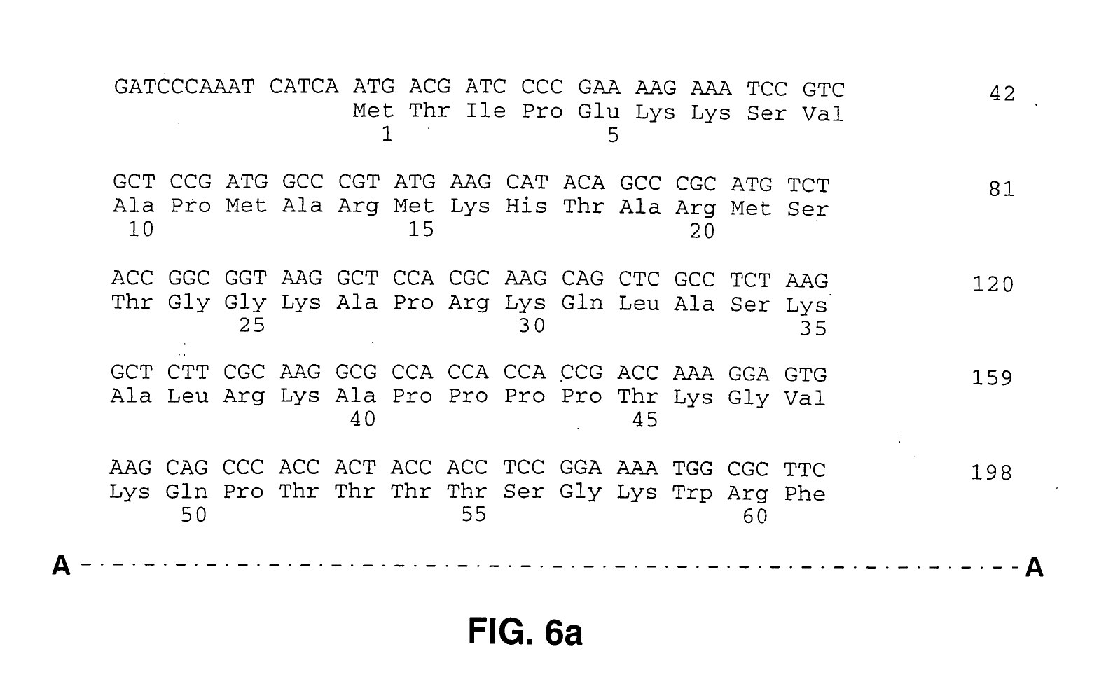

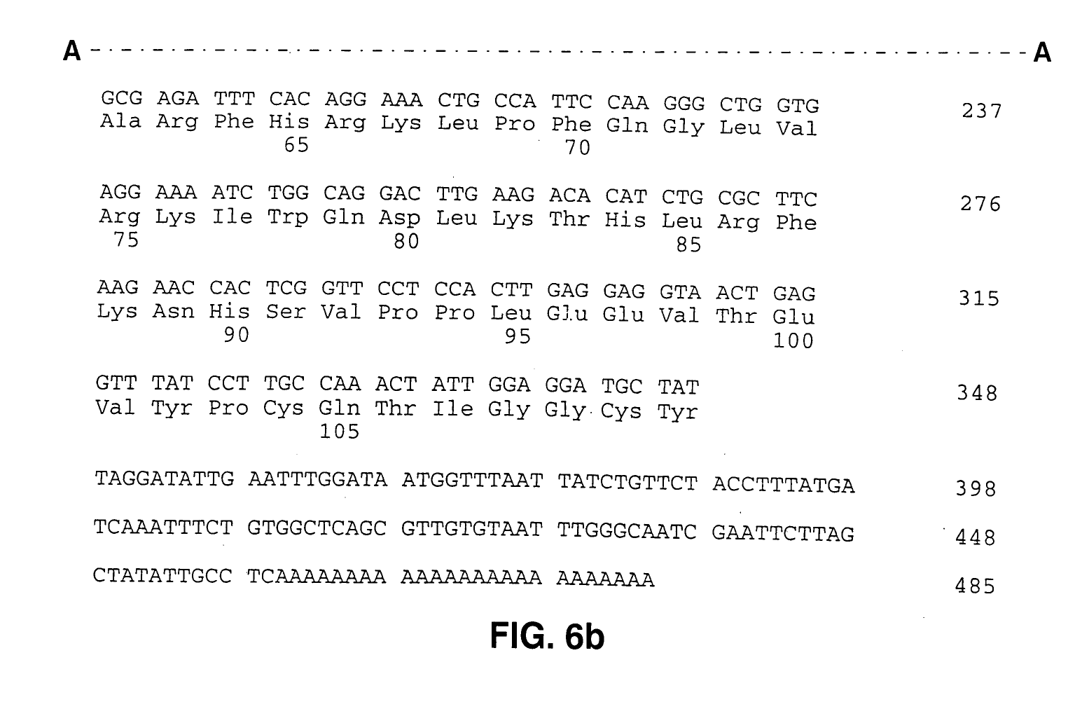

The complete sequence of the gcH3 cDNA clone is shown in FIG. 6. The gcH3 cDNA is of 485 nucleotides and contains a putative open reading frame of 336 bp encoding a protein of 112 amino acids. The predicted gcH3 polypeptide, containing 8% arginine and 12.5% lysine, has a calculated molecular mass of 12.5 kDa. When compared to somatic histone H3, the deduced amino acid sequence of gcH3 exhibits two highly conserved regions located near both terminus of the polypeptide and a variable region of 14 amino acids (position 50 to 64) in the centre region.

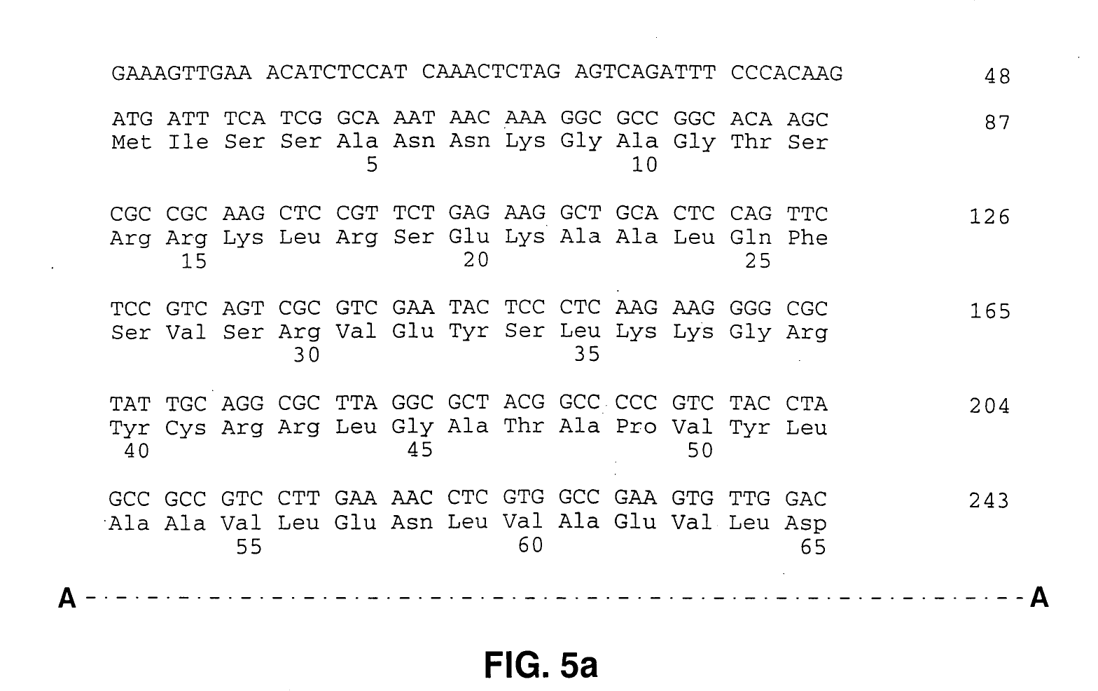

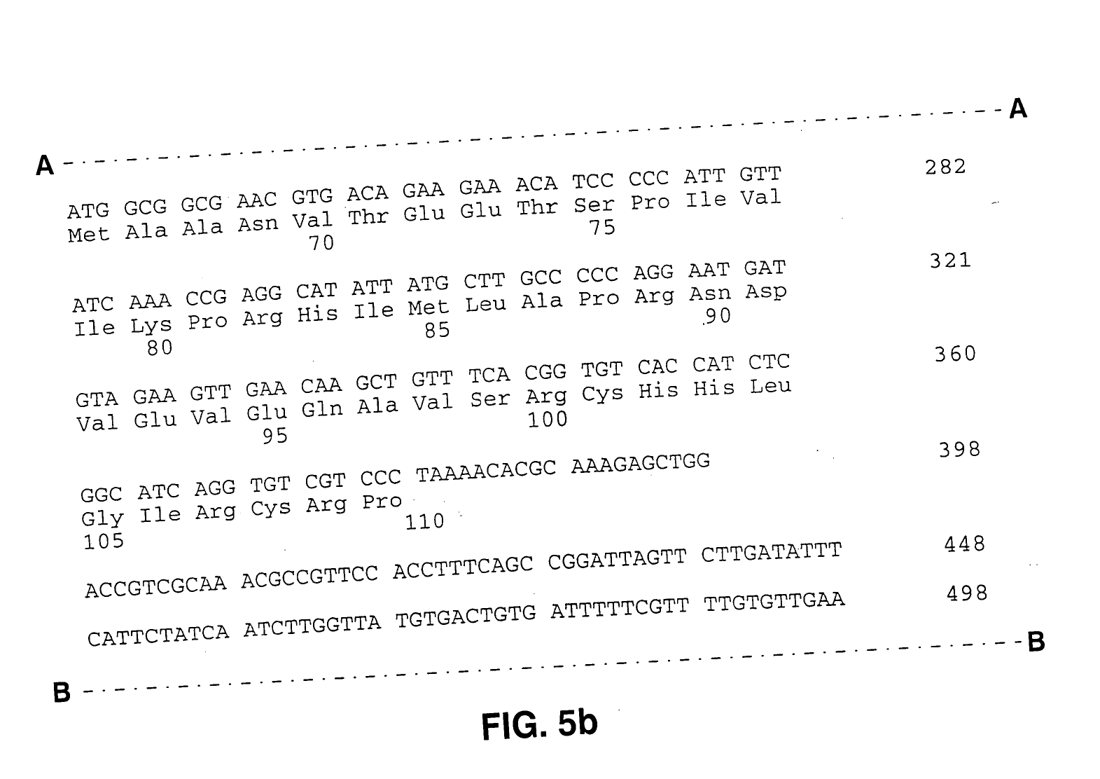

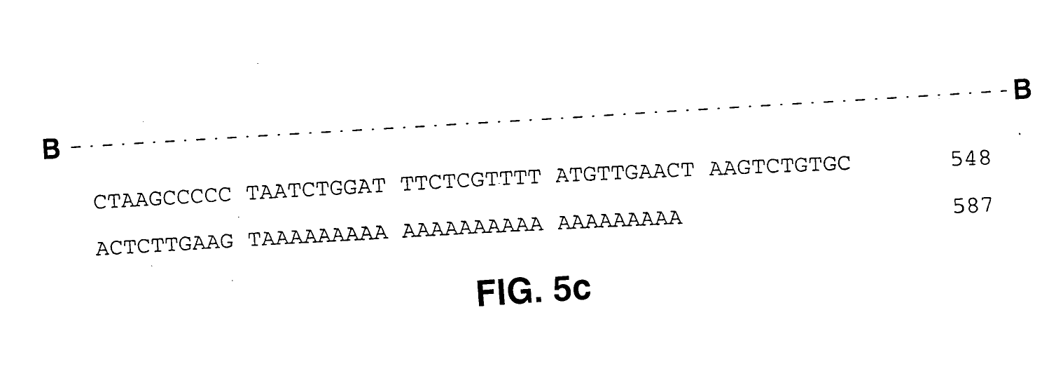

Both gcH2A and gcH3 histone clones were transcribed as polyadenylated mRNAs. Sequencing analysis revealed A/T rich regions resembling the polyadenylation consensus signal and polyadenylated tract bases at their 3′ ends (FIGS. 5 and 6).

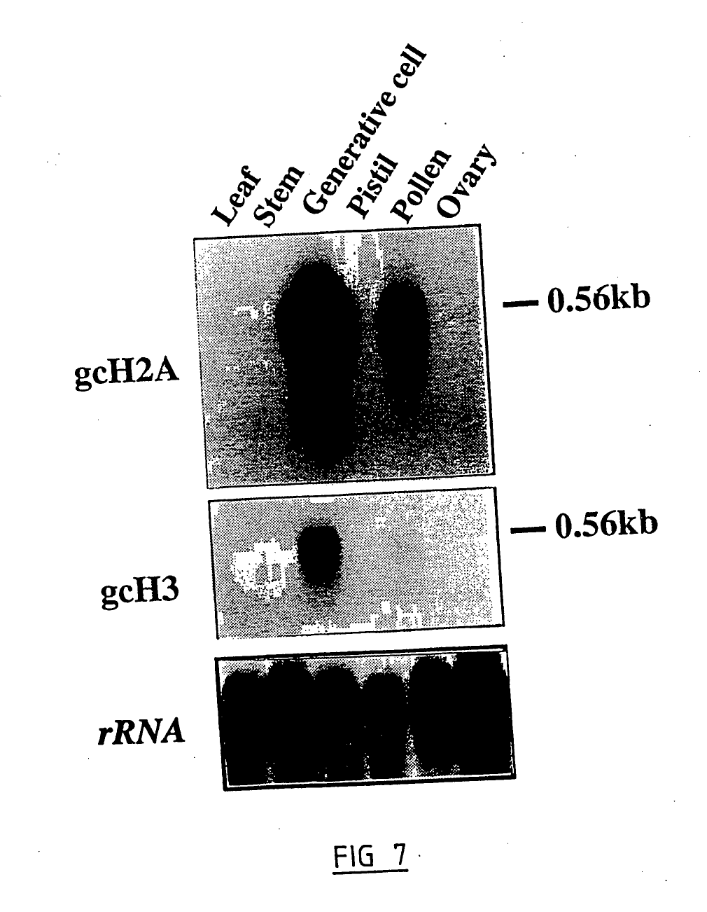

To determine the expression patterns of gcH2A and gcH3, RNA blot analysis was performed with RNA samples from various organs including generative cells, pollen grain, young expanding leaf, stem, pistil and ovary. Considering the highly conserved nature of the histone coding region, hybridization and washing were conducted at high stringency to avoid cross hybridizations with other somatic histone mRNAs. mRNAs corresponding to both gcH2A and gcH3 were detected in generative cells (FIG. 7). A weak hybridization signal was also detected in pollen whereas neither vegetative nor other floral tissues tested showed detectable levels of gcH2A and gcH3 mRNAs. Since pollen grains contain both vegetative and generative cells, it was apparent that the fainter signal detected in pollen RNA was due to the contribution of generative cell only. The inventors tested young leaf and stem tissues from seedlings which have a large number of dividing cells by RNA gel blot as well as RT-PCR analyses. No expression, neither of gcH2A nor of gcH3 was detected. Since the tissues tested represent a broad spectrum of plant organs, it was concluded that both gcH2A and gcH3 are expressed in generative cells only. From the intensity of the hybridization signal, it can be assumed that gcH2A is a highly abundant gene, whereas gcH3 represents a lowly expressed transcript.

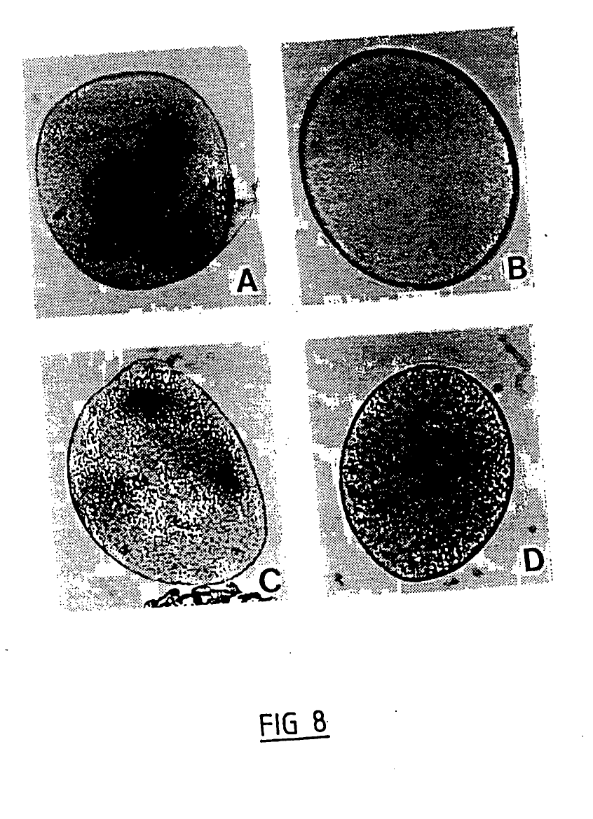

The inventors examined the spatial distribution of gcH2A and gcH3 mRNAs within pollen by in situ hybridization. Digoxigenin (DIG) labelled gcH2A and gcH3 were used to probe whole-mount pollen grains. Accumulation of both gcH2A and gcH3 mRNAs were clearly confined to the generative cell of pollen whereas no hybridization signal was detected in the vegetative cells of pollen (FIGS. 8a, c). No signal was observed in pollen grain probed with control sense probes (FIGS. 8b, d). The accumulation of gcH2A in the generative cell appeared much higher than that of gcH3. The results obtained by in situ hybridization correspond to those of RNA gel blot analysis and clearly demonstrate the generative cell specificity of both gcH2A and gcH3.

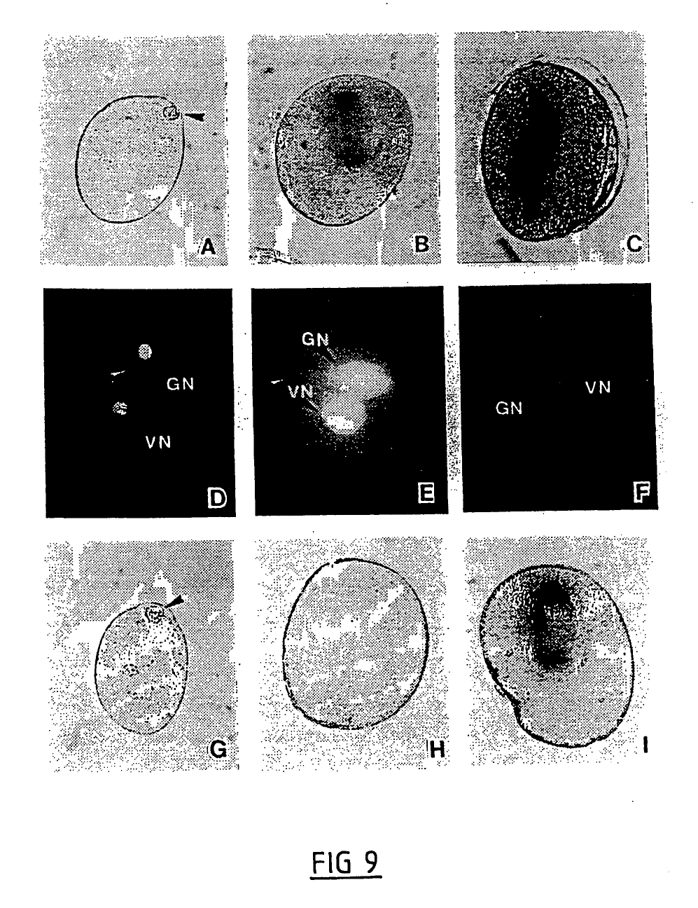

To determine the temporal expression of gcH2A and gcH3, the inventors examined five developmental stages of male gametogenesis. It is well established that three DNA replications occur during male gametogenesis of flowering plants. The first replication occurs prior to meiosis in the microsporocyte or pollen mother cell which produces a tetrad of four haploid microspores. The second replication occurs in the microspore before the first mitotic division (pollen mitosis I) which produces a large vegetative cell and a small generative cell. The third replication takes place in the generative cell before the second mitosis (pollen mitosis II) which results in the formation of two male gametes (sperm cells). To determine whether gcH2A and gcH3 are associated with any of these three DNA replications during male gametogenesis, the inventors performed in situ hybridization in microsporocyte, microspore and three stages of generative cell development. No hybridization signal was observed in pre-meiotic microsporocytes and pre-mitotic microspores. Further, no gcH2A and gcH3 mRNAs were detected in the newly formed generative cell soon after pollen mitosis I (FIGS. 9a, d, g). As development progresses into pollen maturation, the generative cell completely separates from the intine wall of pollen and suspends freely within the vegetative cell cytoplasm. At this stage, the generative cell becomes elongated and spindle-shaped with a large nucleus in the centre and most of its cytoplasm at both ends (FIGS. 9b, e, h). A weak signal was observed at both ends of the generative cell when probing with gcH2A, indicating the initiation of gcH2A mRNA transcription (FIG. 9b). At the time of pollen maturity, the accumulation of gcH2A mRNA in the generative cell reached a very high level as indicated by the strong hybridization signal (FIG. 7c). In comparison to this, the signal obtained with gcH3 probe appeared much weaker (FIG. 7i), and mRNA corresponding to the gcH3 clone could only be detected at the mature stage of pollen development.

### EXAMPLE3

### Cloning of Promoter Region of LGC1

The promoter region of LGC1 was obtained by using the method of Uneven PCR [18]. A gene specific primer and an arbitrary primer were used to generate fragments directly from genomic DNA of lily. Two rounds of PCR amplification were performed.

For the first round of Uneven PCR, a LGC1 gene specific primer (5′-CAGGCATACTTGAATGCTACAAGA-3′ [SEQ ID NO:10]) and an arbitrary 10-mer primer were used. 0.05 μM 10-mer primer, 0.25 μM gene specific primer, 20 ng lily genomic DNA, 200 μM dNTP and 2 units AmpliTaq were added in the 40 μl reaction mix. Cycling conditions of Uneven PCR were 94° C. for 1 min, then for cycle 1, 94° C. for 30 sec, 55° C. for 1 min, 72° C. for 1 min, for cycle 2, 94° C. for 30 sec, 42° C. for 1 min, 72° C. for 1 min; cycle 1 and 2 were repeated 3 times. Then for cycle 7, 94° C. for 15 sec, 57° C. for 30 sec, 72° C. for 30 sec; for cycle 8, 94° C. for 15 sec, 45° C. for 30 sec, 72° C. for 30 sec, cycle 7 and 8 were repeated 20 times. Finally, the sample was held at 72° C. for 5 min. A portion (0.5 μl) of the products from the first round were used as templates for the second round of Uneven PCR. All the components were the same as in the first round except that a nested specific primers (5′-TGTGAACCATACAGAAGAGAACGC-3′ [SEQ ID NO:11 ]) were used to replace the first specific primer. The cycling conditions were: 94° C. for 1 min; then for cycle 1, 94° C. for 15 sec, 57° C. for 30 sec, 72° C. for 30 sec; for cycle 2, 94° C. for 15 sec, 45° C. for 30 sec, 72° C. for 30 sec, cycle 1 and 2 were repeated 20 times; Finally, 72° C. for 5 min.

The samples were size fractionated on 1% w/v agarose gel and blotted on a nylon membrane. The blot was probed with ³²P labelled-LGC1 cDNA. The bands hybridized to LGC1 cDNA were then subcloned into pGEM T-vector. DNA sequencing was performed on both strands by the dideoxy chain-termination method using ABI PRISM™ dye terminator cycle sequencing kit with an automated DNA sequencer.

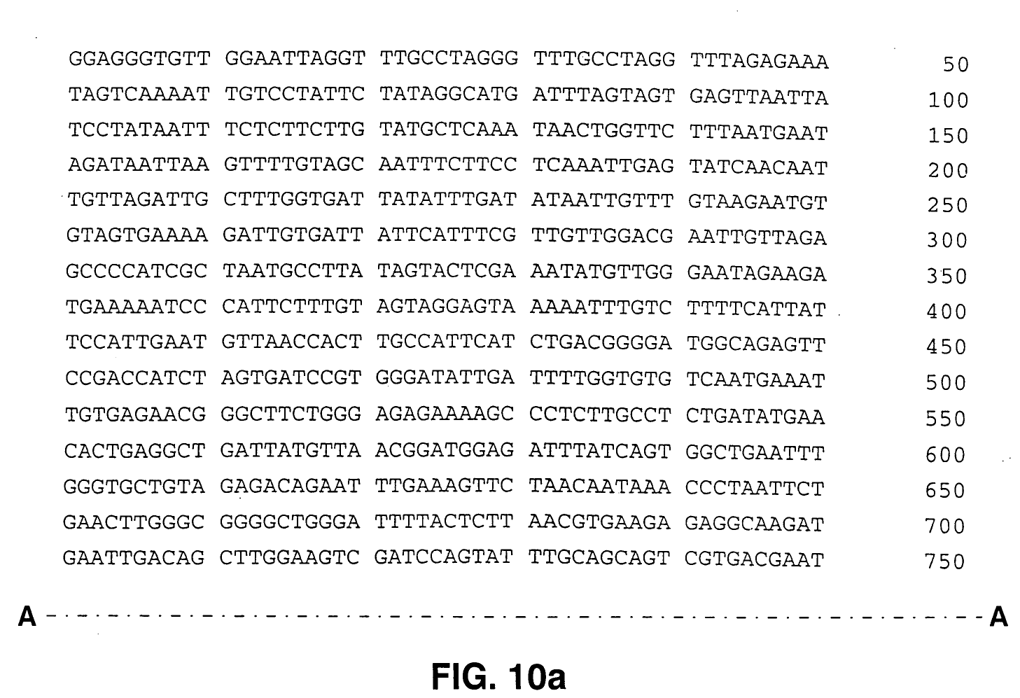

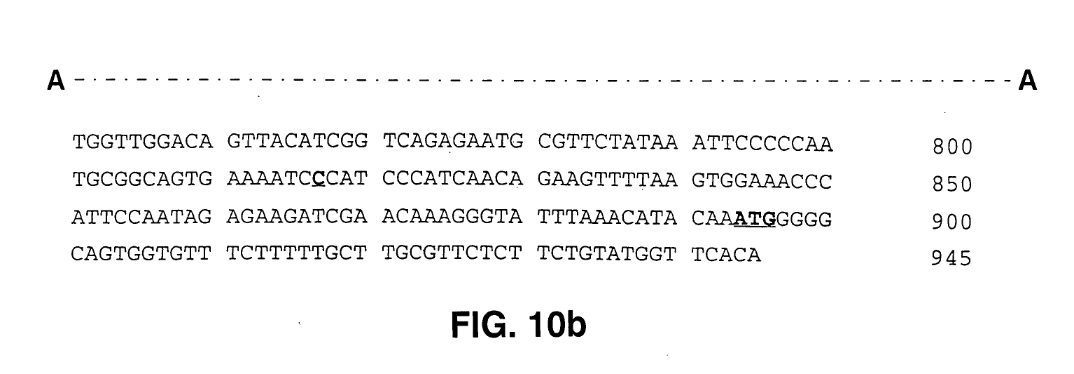

The nucleotide sequence for the LGC1 promoter is shown in SEQ ID NO:9 and in FIG. 10. The transcription start site is nucleotide position 817 and the translation start site (ATG) is nucleotide position 894.

### EXAMPLE 4

### Constructs Comprising the LGC1 Promoter

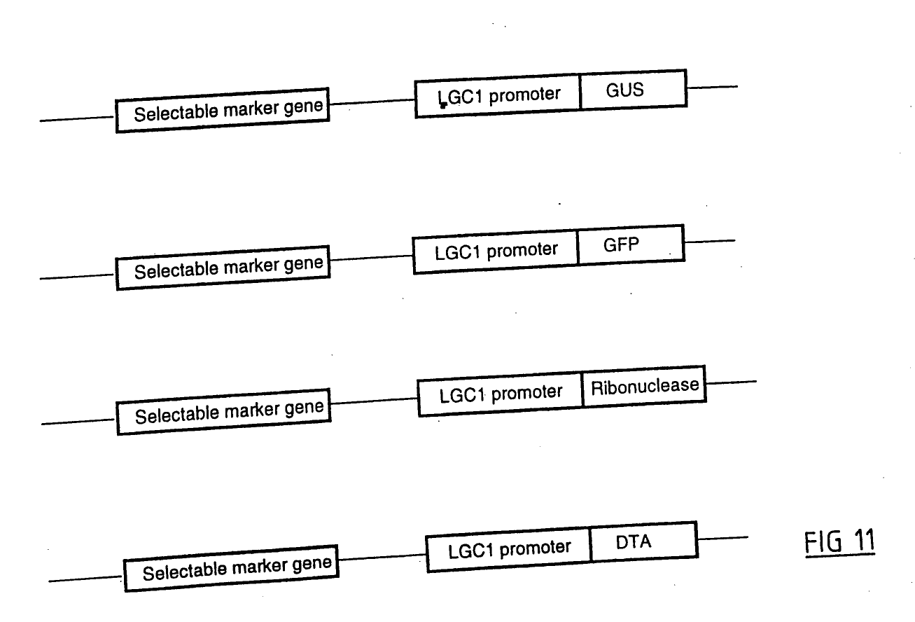

A variety of genetic constructs are made comprising the LGC1 promoter, a nucleotide sequence operably linked thereto and a reporter genetic sequence. Some of these constructs are shown in FIG. 11.

### EXAMPLE 5

### Generative Cell Specific Expression of LGC1 In Transgenic Tobacco

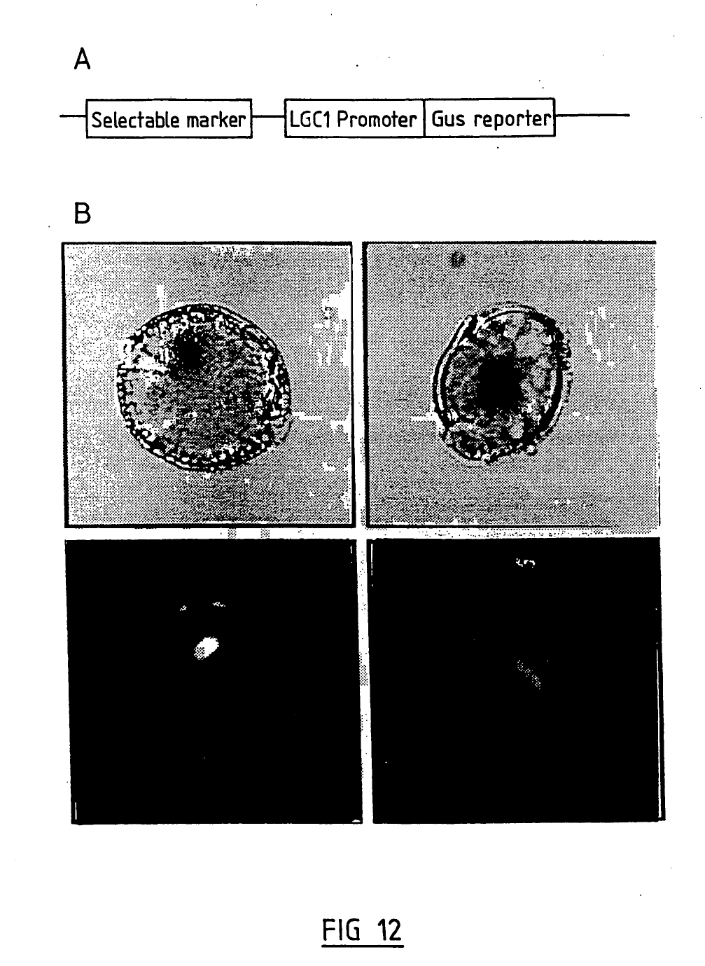

To ascertain that the 5′ non-coding region of LGC1 represents an active promoter and to study its expression pattern, 894 bp of LGC1 upstream sequence were fused to the Escherichia coli β-glucuronidase (Gus) reporter gene (FIG. 12A). The chimaeric fusion construct was introduced into Nicotiana tabacum by Agrobacterium-mediated transformation. Several independent transformants were obtained. Histochemical and fluorimetrical analysis of the transgenic plants for GUS enzyme activity demonstrated that 894 bp flanking region of LGC1 were sufficient to direct gene expression in a generative cell specific manner. None of the transformants showed blue staining in vegetative tissues, like stern, leaf and root, or in different parts of the flower, such as petals, sepals, pistils and ovaries. Counterstaining of mature pollen with DAPI confirmed that Gus gene expression was clearly restricted to the generative cell. The observed activity of the LGC1 5′-flanking region thus reflects the expression of endogenous LGC1 in lily pollen. The results are shown in FIG. 12B.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features. .

### Bibliography

1. Honma, M. A. et al. Proc. Natl. Acad. Sci. USA 90: 6242-6246, 1993.
2. Xu, H. et al. Gene 164: 255-259, 1995.
3. Bouget, F. et al. J. Phycol. 31: 1027-1030, 1995.
4. Bouget, F. et al. Plant Cell 8: 189-201, 1996.
5. Torres, M et al. Plant J. 8: 317-321, 1995.
6. Blomstedt, C. K. et al. Plant Mol. Biol. 31: 1083-1086, 1996.
7. Osley, M. A. Annu. Rev. Biochem. 60: 827-, 1991.
8. Tanaka, I. Protoplasma 142: 68-73, (1988)
9. Sanger, F. et al. Proc. Natl. Acad. Sci. USA 74: 5463-5467, 1977.
10. Chomczynski, P. et al. Anal. Biochem. 162: 156-159, 1987.
11. Bouget, F. et al. J. Phycol. 31: 1027-1030, 1995.
12. Terres, M. A. et al. Plant J. 8: 317-321, 1995.
13. Bouget, F. et al. Plant Cell 8: 189-201, 1996.
14. Huh, H. G. et al. Plant Mol. Biol. 33: 791-802, 1997.
15. Rommens, C. M. T. et al. Mol. Gene. Genet. 231: 433-441, 1992.
16. Roberts, M. In Plant Gene Isolation: Principles and Practice, Ed. by C. D. Foster and D. Twell, pp 301-328, 1996, John Wiley & Sons Ltd.
17. McCormick, S. Plant Cell 5: 1265-1275, 1993.
18. Chen, X and Wu, R Gene 185: 195-199, 1997.
19. Marmur, J and Doty, P. J. Mol. Biol. 5: 109, 1962.
20. Bonner, W. M. and Laskey, R. A. Eur. J. Biochem. 46: 83, 1974.

1. An isolated nucleic acid molecule comprising a nucleotide sequence or a complementary nucleotide sequence corresponding to a gene or derivative thereof or a region of said gene facilitating its expression wherein said gene is specifically expressed in generative cells and sperm cells of a plant but wherein said gene does not encode a histone.

2. An isolated nucleic acid molecule according to claim 1 wherein said plant is selected from a legume, crop plant, cereal plant, a grass, a fruiting plant and a flowering plant.

3. An isolated nucleic acid molecule according to claim 2 wherein the plant is a lily or a related plant.

4. An isolated nucleic acid molecule according to claim 3 comprising a nucleotide sequence which encodes an amino acid sequence selected from SEQ ID NO:4, SEQ ID NO:6 and SEQ ID NO:8 or an amino acid sequence having at least 40% identity to any one of SEQ ID NO:4, SEQ ID NO:6 or SEQ ID NO:8.

5. An isolated nucleic acid molecule according to claim 4 comprising a nucleotide sequence selected from the group consisting of SEQ ID NO:3, SEQ ID NO:5 and SEQ ID NO:7 or a nucleotide sequence having at least 50% identity to any one of SEQ ID NO:3, SEQ ID NO:5 or SEQ ID NO:7 or is a nucleotide sequence capable of hybridizing to any one of SEQ ID NO:3, SEQ ID NO:5 or SEQ ID NO:7 under low stringency conditions at 42° C.

6. An isolated nucleic acid molecule according to claim 1 wherein said nucleic acid molecule is a promoter or a functional derivative which directs plant generative cell and sperm cell specific expression.

7. An isolated nucleic acid molecule according to claim 6 comprising a nucleotide sequence or complementary nucleotide sequence which is capable of hybridizing under low stringency conditions at 42° C. to a genomic region encompassing at least about 2 kbp upstream of the genomic nucleotide sequence corresponding to any one of SEQ ID NO:3 or SEQ ID NO:5 or SEQ ID NO:7.

8. An isolated nucleic acid molecule according to claim 6 comprising a nucleotide sequence or complementary nucleotide sequence substantially as set forth in SEQ ID NO:9 or a nucleotide sequence capable of hybridizing thereto under low stringency conditions at 42° C. or a nucleotide sequence having at least 50% identity to SEQ ID NO:9.

9. An isolated nucleic acid molecule comprising a nucleotide sequence or complementary nucleotide sequence substantially as set forth in SEQ ID NO:9 or a nucleotide sequence capable of hybridizing thereto under low stringency conditions at 42° C. or a nucleotide sequence having at least 50% identity to SEQ ID NO:9 and wherein said nucleic acid molecule is capable of directing plant generative cell and sperm cell specific expression of a nucleotide sequence operably linked thereto.

10. An isolated nucleic acid molecule according to claim 9 wherein the nucleotide sequence operably linked to the nucleic acid molecule encodes or defines GUS, GFP, a ribonuclease, DTA, an antisense molecule, a transposon or a lethal gene.

11. A method of inducing or otherwise facilitating male sterility in a plant, said method comprising operably linking a cytotoxic nucleic acid molecule to a promoter which directs plant generative cell and sperm cell specific expression in said plant such that upon direction by said promoter, the cytotoxic nucleic acid molecule is expressed to produce a product which inactivates, kills or otherwise renders substantially non-functional generative cells and/or sperm cells in said plant wherein said promoter is not a histone gene-specific promoter.

12. A method according to claim 11 wherein said plant is a legume, crop plant, cereal plant, a grass, a fruiting plant and a flowering plant.

13. A method according to claim 11 wherein the cytotoxic nucleic acid molecule encodes or comprise a cytotoxic protein, an antisense molecule to a particular gene, a ribozyme or a plantabody.

14. A method according to claim 11 wherein the promoter corresponds to a nucleotide sequence which hybridizes under low stringency conditions to a genomic region comprising at least about 2 kbp upstream of a gene corresponding to any one of SEQ ID NO:3, SEQ ID NO:5 or SEQ ID NO:7.

15. A method according to claim 14 wherein the promoter comprises a nucleotide sequence substantially as set forth in SEQ ID NO:9 or a nucleotide sequence capable of hybridizing thereto under low stringency conditions at 42° C. or a nucleotide sequence having at least 50% identity to SEQ ID NO:9.

16. A genetic construct comprising a generative cell and sperm cell specific promoter operably linked to a transposase gene, said transposase gene capable of inducing transposition of a transposable element such that upon expression of said promoter, the transposase gene is expressed facilitating transposition of said transposable element wherein said promoter is not a histone gene-specific promoter.

17. A genetic construct according to claim 16 wherein where the promoter comprises a nucleotide sequence substantially as set forth in SEQ ID NO:9, or a nucleotide sequence capable of hybridizing thereto under low stringency conditions at 42° C. or a nucleotide sequence having at least 50% identity to SEQ ID NO:9.

18. A genetic construct according to claim 16 wherein the transposase gene is the activator (Ac) transposase.

19. A male sterile plant generated by the method of claim 11.

20. A male sterile plant according to claim 19 which provides seedless fruit or fruit with reduced seed content.


SEQUENCE LISTING (11 sequences)

SEQ ID NO: 1 - DNA, 24 bp, Artificial Sequence
  Description of Artificial Sequenceprimer
gtactcttaagcatacaacatgag

SEQ ID NO: 2 - DNA, 24 bp, Artificial Sequence
  Description of Artificial Sequenceprimer
caggcatacttgaatgctacaaga

SEQ ID NO: 3 - DNA, 625 bp, Lilium longiflorum
  CDS (82)..(468)
gccatcccatcaacagaaggtttaagtggaaatccatttcattagaaaagatcggacaaagggtactcttaagcatacaacatgagggcggtggcggttttctttgcttgcgttctcttctgtatggttcacaaagccgcacttgcggatgataaaacgtgcaaccctacagattttatggttacccaaaccataactggattgacaatcggcggtaaacaagagttcgaggtcaatttaataaacaatttgtattgtgcacaatctaatgtcaaagtttcatgtgacgggcttcataccaccgaaccaatagatcctcacattatcagaccacttagtgacggaacgaacaactgccttgtcaacaatggagcgcctatttctcatgctactcttgtagcattcaagtatgcctgggatgttcctccatctttcagcatcatcagctctgatataaattgctcctaaggagaaaattctagttggcagagaataatcatatagtcttttttactgagctatttaattttttcaattttcaccaataagattattttaatggaatgttaatgtattagaattgaaaaataaaaaaaaaaaaaaaaaaaaaaaaaaaaaaaaaaaa

SEQ ID NO: 4 - protein, 128 aa, Lilium longiflorum
MRAVAVFFACVLFCMVHKAALADDKTCNPTDFMVTQTITGLTIGGKQEFEVNLINNLYCAQSNVKVSCDGLHTTEPIDPHIIRPLSDGTNNCLVNNGAPISHATLVAFKYAWDVPPSFSIISSDINCS

SEQ ID NO: 5 - DNA, 587 bp, Lilium longiflorum
  CDS (49)..(378)
gaaagttgaaacatctccatcaaactctagagtcagatttcccacaagatgatttcatcggcaaataacaaaggcgccggcacaagccgccgcaagctccgttctgagaaggctgcactccagttctccgtcagtcgcgtcgaatactccctcaagaaggggcgctattgcaggcgcttaggcgctacggcccccgtctacctagccgccgtccttgaaaacctcgtggccgaagtgttggacatggcggcgaacgtgacagaagaaacatcccccattgttatcaaaccgaggcatattatgcttgcccccaggaatgatgtagaagttgaacaagctgtttcacggtgtcaccatctcggcatcaggtgtcgtccctaaaacacgcaaagagctggaccgtcgcaaacgccgttccacctttcagccggattagttcttgatatttcattctatcaatcttggttatgtgactgtgatttttcgttttgtgttgaactaagccccctaatctggatttctcgttttatgttgaactaagtctgtgcactcttgaagtaaaaaaaaaaaaaaaaaaaaaaaaaaaa

SEQ ID NO: 6 - protein, 110 aa, Lilium longiflorum
MISSANNKGAGTSRRKLRSEKAALQFSVSRVEYSLKKGRYCRRLGATAPVYLAAVLENLVAEVLDMAANVTEETSPIVIKPRHIMLAPRNDVEVEQAVSRCHHLGIRCRP

SEQ ID NO: 7 - DNA, 485 bp, Lilium longiflorum
  CDS (16)..(348)
gatcccaaatcatcaatgacgatccccgaaaagaaatccgtcgctccgatggcccgtatgaagcatacagcccgcatgtctaccggcggtaaggctccacgcaagcagctcgcctctaaggctcttcgcaaggcgccaccaccaccgaccaaaggagtgaagcagcccaccactaccacctccggaaaatggcgcttcgcgagatttcacaggaaactgccattccaagggctggtgaggaaaatctggcaggacttgaagacacatctgcgcttcaagaaccactcggttcctccacttgaggaggtaactgaggtttatccttgccaaactattggaggatgctattaggatattgaatttggataatggtttaattatctgttctacctttatgatcaaatttctgtggctcagcgttgtgtaatttgggcaatcgaattcttagctatattgcctcaaaaaaaaaaaaaaaaaaaaaaaaa

SEQ ID NO: 8 - protein, 111 aa, Lilium longiflorum
MTIPEKKSVAPMARMKHTARMSTGGKAPRKQLASKALRKAPPPPTKGVKQPTTTTSGKWRFARFHRKLPFQGLVRKIWQDLKTHLRFKNHSVPPLEEVTEVYPCQTIGGCY

SEQ ID NO: 9 - DNA, 945 bp, Lilium longiflorum
ggagggtgttggaattaggtttgcctagggtttgcctaggtttagagaaatagtcaaaattgtcctattctataggcatgatttagtagtgagttaattatcctataatttctcttcttgtatgctcaaataactggttctttaatgaatagataattaagttttgtagcaatttcttcctcaaattgagtatcaacaattgttagattgctttggtgattatatttgatataattgtttgtaagaatgtgtagtgaaaagattgtgattattcatttcgttgttggacgaattgttagagccccatcgctaatgccttatagtactcgaaatatgttgggaatagaagatgaaaaatcccattctttgtagtaggagtaaaaatttgtcttttcattattccattgaatgttaaccacttgccattcatctgacggggatggcagagttccgaccatctagtgatccgtgggatattgattttggtgtgtcaatgaaattgtgagaacgggcttctgggagagaaaagccctcttgcctctgatatgaacactgaggctgattatgttaacggatggagatttatcagtggctgaatttgggtgctgtagagacagaatttgaaagttctaacaataaaccctaattctgaacttgggcggggctgggattttactcttaacgtgaagagaggcaagatgaattgacagcttggaagtcgatccagtatttgcagcagtcgtgacgaattggttggacagttacatcggtcagagaatgcgttctataaattcccccaatgcggcagtgaaaatcccatcccatcaacagaagttttaagtggaaacccattccaatagagaagatcgaacaaagggtatttaaacatacaaatgggggcagtggtgtttctttttgcttgcgttctcttctgtatggttcaca

SEQ ID NO: 10 - DNA, 24 bp, Artificial Sequence
  Description of Artificial Sequenceprimer
caggcatacttgaatgctacaaga

SEQ ID NO: 11 - DNA, 24 bp, Artificial Sequence
  Description of Artificial Sequenceprimer
tgtgaaccatacagaagagaacgc